(19) United States
(12) Patent Application Publication
(10) Pub. No.: US 2008/0189045 A1
(43) Pub. Date: Aug. 7, 2008

(54) Method for the Collection And Distribution of Cord Blood Stem Cells

(76) Inventor: Thomas E. Moore, Tucson, AZ (US)

Correspondence Address: PILLSBURY WINTHROP SHAW PITTMAN LLP, P.O. BOX 10500, MCLEAN, VA, 22102, US

(21) Appl. No.: 11/671,967

(22) Filed: Feb. 6, 2007

(51) Int. Cl. G01N 33/48

(52) U.S. Cl. 702/19

(57) ABSTRACT

The present invention provides a method for the collection and distribution of cord blood stem cells, particularly in order to increase the number of usable cord blood stem cells that are collected overall. By using a single collection and distribution entity that applies a uniform protocol to obtain cord blood stem cell samples at each of a plurality of different collection facilities, a greater number of samples for both private and public cord blood stem cell banks can be obtained.

### BACKGROUND OF THE INVENTION

1. Field of the Invention

The present invention provides a method for the collection and distribution of cord blood stem cells.

2. Field of the Related Art

There are currently four modalities of cord blood collection and storage, each of which is described below.

With the first, “family” cord blood banking—cord blood is collected from the baby for primary use by the child and 1 st and 2nd degree relatives. The family pays the bank for processing and storage of the CB sample. For profit companies operate the banks and, in the case of the larger banks, collect cord blood on a national scale using a network of collecting physicians, hospitals, and field representatives. Mothers are made aware of this option through consumer and professional channel marketing. The collected cord blood sample is the property of the family.

With the second, “public” cord blood banking—cord blood is collected for processing and storage in an anonymous bank. Samples are used in an allogeneic setting and require donor/host genetic matching prior to clinical use. Public banks are not for profit institutions, supported largely by NIH grants, and operate in a small number of regional hospitals proximate to the bank itself. Mothers are made aware of this option at the time of birth, or shortly before, and the cord blood is collected by staff members who are typically direct staff of the public bank and resident at the regional hospital. There is a limited ability to collect cord blood with specific characteristics, such as sample size, ethnic background, family health history, etc. owing to the limited hospital/donor reach and information window available. The collected cord blood sample is the property of the public bank.

With the third, known as a Designated Transplant Banking (DTB)—cord blood is collected from the baby for primary use by a 1st degree relative already identified with a disease for which the baby's cord blood stem cells may provide a viable therapeutic option. This modality is offered by CBR Systems, Inc. There is no charge to the family for this service. The cord blood sample is typically the property of the DTB.

With the fourth, known as Emergency Cord Blood Banking (also known as Low APGAR Collection and various other names)—cord blood is collected from the baby based on a metric determined at the time of birth by the physician, such as a low APGAR score, or other metric which may be predictive of a condition for which the collected stem cells may be of therapeutic value for the child. There is a nominal charge to the family. The cord blood sample is the property of the “Family” bank for a period of time and can then revert to the parents in a “conversion” to family banking.

Current federal regulations restrict Family banks from operating as Public banks and Public banks are restricted from Family banking by charter, funding sources, and an inability to be competitive in Family banking. This results in substantial inefficiencies on both sides. The Family banks can not leverage their highly efficient, high volume, collection and processing systems to lower the per sample cost of publicly banked samples, and the public banks are forced into a highly inefficient collection system involving direct staff at limited regional hospitals. The public banks are also constrained relative to the characteristics of the cord blood they can collect as discussed above.

It is also taught, as described in U.S. Pat. No. 5,993,387 entitled “Computer-based mixed-use registry of placental and umbilical cord stem cells,” assigned to the present assignee, that it would be advantageous to have a cord blood stem cell bank established that allows for both typed, anonymous samples such as found in Public banks and for Family Banking that allows for exclusive family use and retention of the cord stem cell samples. While this mixed use bank system provides a mechanism for a single cord blood bank that can process and store samples, the described mixed use bank has never met with any significant acceptance, and also did not provide any mechanism to increase the number of cord blood stem cell samples collected.

### SUMMARY OF THE INVENTION

The present invention provides a method for the collection and distribution of cord blood stem cells.

In one aspect, the present invention provides a mechanism to increase the number of usable cord blood stem cells that are collected.

In a particular aspect, the present method provides a method of collecting and distributing cord blood stem cells prior to processing and storing in which a plurality of cord blood stem cell banks are identified that each have requirements for various different cord blood stem cell samples, the plurality of cord blood stem cell banks including at least one private stem cell bank and at least one public stem cell bank. A plurality of facilities that will obtain cord blood stem cell samples are identified using a uniform protocol. The uniform protocol is then used to obtain cord blood stem cell samples at each of the plurality of facilities, and includes (1) providing, if a particular cord blood stem cell sample was previously identified for private storage at the at least one private stem cell bank, the particular cord blood stem cell sample to the at least one private stem cell bank for processing and storage; and (2) determining, if the particular cord blood stem cell sample was not previously identified for private storage, whether characteristics of the mother or the particular cord blood stem cell sample match predetermined characteristics for cord blood stem cell samples required by the at least one public stem cell bank. The particular cord blood stem cell sample is then supplied to the at least one public stem cell bank for processing and storage if the characteristics of the mother or the particular cord blood stem cell sample match predetermined characteristics for cord blood stem cell samples required by the at least one public stem cell bank.

In another aspect, the present invention provides a networked computer system that allows for the collection and distribution of cord blood stem cells.

In a particular aspect, there is provided a method of collecting and distributing cord blood stem cells prior to processing and storing in which a plurality of cord blood stem cell banks that each have requirements for various different cord blood stem cell samples are identified, the plurality of cord blood stem cell banks including at least one private stem cell bank and at least one public stem cell bank. A plurality of facilities that will obtain cord blood stem cell samples using a uniform protocol are also identified. The uniform protocol is then used to obtain cord blood stem cell samples at each of the plurality of facilities, the uniform protocol includes (1) providing a networked computer system that includes a cord blood capture computer program, such that at each of the plurality of facilities there exists a computer that can execute the cord blood capture computer program; (2) marking, using the cord blood capture computer program, if a particular cord blood stem cell sample was previously identified for private storage at the at least one private stem cell bank, the marking including matching the particular cord blood stem cell sample with a code, and applying that code to an entry associated with the cord blood capture computer program that designates that particular cord blood stem cell sample with the at least one private stem cell bank; (3) determining, if the particular cord blood stem cell sample was not previously identified for private storage, whether characteristics of the mother or the cord blood stem cell sample match predetermined characteristics for cord blood stem cell samples required by the at least one public stem cell bank; and (4) marking, using the cord blood capture computer program and if the characteristics of the particular cord blood stem cell sample matched the predetermined characteristics, the particular cord blood sample for public storage at the at least one public stem cell bank, the marking including matching the particular cord blood stem cell sample with another code, and applying that another code to an entry associated with the cord blood capture computer program that designates that particular cord blood stem cell sample with the at least one public stem cell bank.

### BRIEF DESCRIPTION OF THE DRAWINGS

These and other aspects and features of the present invention will become apparent to those of ordinary skill in the art upon review of the following description of specific embodiments of the invention in conjunction with the accompanying figures, wherein:

FIG. 1 illustrates a method according to the present invention; and

FIG. 2 illustrates a networked system according to the present invention.

### DETAILED DESCRIPTION OF THE PREFERRED EMBODIMENTS

The invention disclosed herein seeks to increase the number of usable cord blood stem cells that are collected. As described, this increase is achieved by implementing a uniform protocol that can be used by both Public stem cell banks and Family stem cell banks, with a single entity that implements the collection of stem cell cord blood samples, and their distribution to different stem cell banks. Through implementation of this uniform protocol by a single entity, this allows for a dramatic increase in the scale and sample specificity in various different cord blood stem cell banking systems (Public, DTB, emergency) by leveraging existing features of the Family banking system, which include its existence on a national scale, its usage of mother education, and the attention of the Family banking system to lead time in collection, as well as its existing channel of professional physicians who use it.

By providing a single entity, which can function using a networked computer system with the internet and other channels, based upon the uniform protocol, a mother and father can choose from among all the possible modalities for cord blood collection and banking available.

As an overview, a mother and father interested in cord blood collection and banking are made aware of all options available through the collection and distribution entity's various educational offerings, including its website(s), direct mail, advertising, email programs, and professional channels. If a mother and father choose Family, DTB, or Emergency banking, collection and distribution entity's handles the overall process in a vertically integrated fashion; providing education, registration, data input, collection, shipping (through a third party), testing, processing and, if needed, banking.

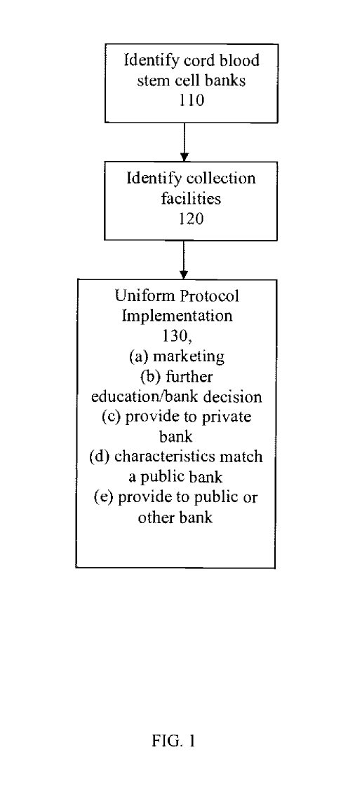

As shown in FIG. 1, in one aspect, the present method provides a system for collecting and distributing cord blood stem cells prior to processing and storing them in a bank. As shown by step 110, a plurality of cord blood stem cell banks are identified that each have requirements for various different cord blood stem cell samples. This plurality of cord blood stem cell banks including at least one private stem cell bank and at least one public stem cell bank, and may also have an emergency bank and a DTB bank, and will preferably have a considerable number of different public stem cell banks associated with it. As shown by step 120, a plurality of facilities that will obtain cord blood stem cell samples are then, identified, and, in step 130 agreements reached with each such that each will use a uniform protocol with respect to obtaining cord blood stem cell samples. The uniform protocol is then used to enroll donors, obtain information from the donors and their family, and determine which one of the various types of cord blood banks to use, in order to then ultimately obtain cord blood stem cell samples from the donor at one of the plurality of facilities, and then forward the collected sample to the appropriate one of the cord blood stem cell banks. The uniform protocol step this includes step 130(a) marketing and educating by outreach programs (either internet/web based, direct mail, professional referrals, advertising, email, or other educational programs, the services of the various cord blood bank types. In step 130(b) there is the step of further educating each particular individual on options available to that individual, so that a choice can be made which one of the various different banks to use. In step 130(c), if a private cord blood bank was chosen (including a DTB bank), then donor information is obtained, enrollment materials provided and executed, and ultimately at birth of the donor, obtaining and providing, if a particular cord blood stem cell sample was previously identified for private storage, the particular cord blood stem cell sample to the selected one private stem cell bank for processing and storage. If a public bank was chosen, then step 130(d) follows, with initially there being the need to obtain personal information as well as information needed to fulfill requirements of the particular public bank. When this is information is obtained (either prior to or immediately after obtaining the particular cord blood stem cell sample if that is necessary) characteristics of the mother or the particular cord blood stem cell sample are used to determine if they match predetermined characteristics for cord blood stem cell samples required by a particular public stem cell bank. If those characteristics match, then, in step 130(e), the particular cord blood stem cell sample that was obtained is then supplied to the at least one public stem cell bank for processing and storage.

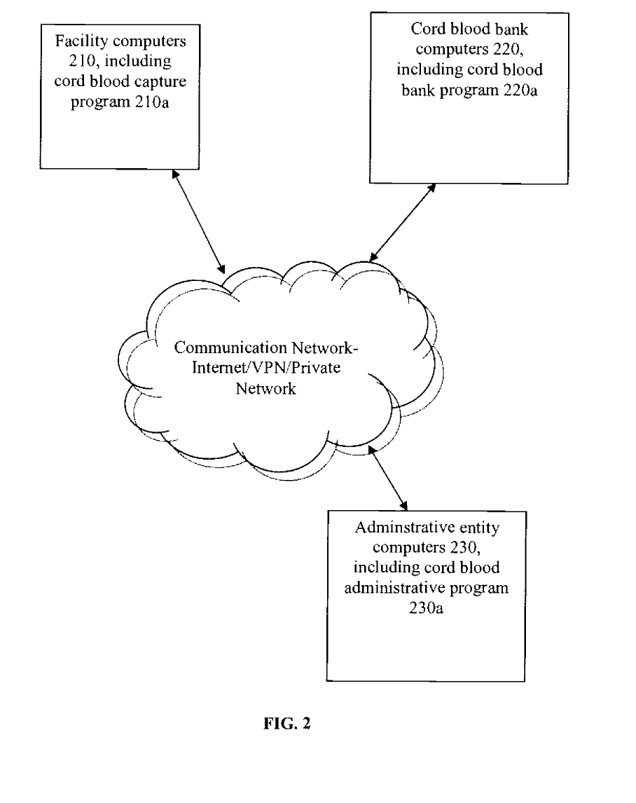

FIG. 2 illustrates a networked computer system 200 that includes various computers 210 at different cord blood collection and educational facilities, computers 220 at different cord blood banks, and also includes computer(s) 230 at the entity that implements the collection of stem cell cord blood samples, as described above. The computers 210 will contain a cord blood information capture computer program 210a, such that at each of the plurality of facilities there exists a computer 210 that can execute the cord blood capture computer program 210a that will assist in gathering information about the mother, father, child and the cord blood stem cells. The computers 220 will include a cord blood bank program 220a and are typically used by the cord blood banks to track the cord blood that is stored at that facility, as well as track the requirements that are being required by the facility (particularly in the case of the public banks).

The cord blood information capture computer program 210a will also preferably include the ability to allow private marking to indicate if a particular cord blood stem cell sample was previously identified for private storage at a private stem cell bank. The private marking will allow for matching the particular cord blood stem cell sample with a code, and applying that code to an entry associated with the cord blood capture computer program that designates that particular cord blood stem cell sample with the private stem cell bank.

The cord blood information capture computer program 210a will also preferably include the ability to allow public marking to indicate if a particular cord blood stem cell sample was previously identified for public storage at a public stem cell bank. The public marking will allow for matching the particular cord blood stem cell sample with another code, and applying that code to an entry associated with the cord blood capture computer program that designates that particular cord blood stem cell sample with the public stem cell bank.

The computer(s) 230 typically run the administrative software 230a that allows for the collection of the information from the computers 210 relating to the cord blood sample that has been or will be collected, as well as the data from the computers 220 relating to the type of cord blood desired by different facilities, in order to then run a software program that contains the functions described herein that correlate the information from the cord blood that has been or will be collected with the data relating to the type of cord blood desired by different facilities in order to determine if there is a match. This is typically implemented using a database 240. In particular, the software program 230a that provides for matching, either in advance of a birth based upon information, preferably from information on the mother, or at birth based upon information from the particular stem cell sample, or both, are preferably stored in the database 240.

The database 240 can have many different configurations. In one configuration, each record of the database will have a donor identifier field, a sample type information field 120 and a sample identifier code field. Other fields, and sub-fields, are also possible. Preferably the database can be searched on at least any of the fields or sub-fields. Further, it is preferred for that the sample type information field has genetic and other searchable type information about the cord stem cell sample for the donor associated with the record. The field will also preferably contain information about other family members, particularly the mother, as described herein, which information can also include known disease histories of the donor's family. The type information can also include such information as donor's family medical history and information relating to ethnic and geographic origin of the donor or donor family. Other donor information such as donor phenotype may also be included in the type information.

Each database record will also have a field indicating where the sample associated with the record will be delivered for storage, and will typically have sub-fields relating to the storage bank requirements (such as the particular program to which the sample will be stored in a public use bank). Each database record will also preferably have a field indicating the facility from which the sample was obtained, as well as sub-fields relating to specifics on the obtaining of the sample (such as the physician/clinician name who obtained the sample).

It is also noted that for privacy reasons, not all users of the database are provided with access to any or all of the fields. Therefore, for example, only some users can search based on the type information field and only restricted access is provided to searches on the sample identifier field. In this manner, unauthorized matching of type information with donors is prevented.

Communications between these computers 210, 220 and 230 (which can be run as servers, and may also include various different types of networks) will typically be through secure communication channels, such as virtual private networks over the internet, but may also include other types of networks. It is also understood that the functions of computers 210, 220 and 230 as described above can be implemented from a central server facility, or using computers that have different combinations of these functions.

In a particular aspect, using the networked computer system and the various banking facilities with the uniform protocol, the collection and distribution entity can work with a mother and father who desire to choose Public banking to determine if there is a public bank that is, at that time, and in advance of the actual birth of the child, looking for samples that are of the type that will result from the birth of the child by that mother and father. In particular, the collection and distribution entity can determine whether a particular Public bank is looking for samples of the type that correspond to that which will result, and then provide information to the Public bank on the sample that will be obtained. As a result, by targeting a specific ethnicity, family history, sample size (based on data acquired in the window between sign up and collection), etc. to the Public bank, the Public bank ends up with a much greater possible range of potential donors (rather than only the small donor number that would otherwise normally enter that particular Public bank), and thereby a larger volume of samples at an overall lower collection cost. The collection and distribution entity, in this aspect, preferably provides all services through the shipping of the sample, after which the receiving bank would then test, process and bank the actual sample.

Although the present invention has been particularly described with reference to embodiments thereof, it should be readily apparent to those of ordinary skill in the art that various changes, modifications and substitutes are intended within the form and details thereof, without departing from the spirit and scope of the invention. Accordingly, it will be appreciated that in numerous instances some features of the invention will be employed without a corresponding use of other features. Further, those skilled in the art will understand that variations can be made in the number and arrangement of components illustrated in the above figures. It is intended that the scope of the appended claims include such changes and modifications.

1. A method of collecting and distributing cord blood stem cells prior to storing, the method comprising the steps of:
identifying a plurality of cord blood stem cell banks that each have requirements for various different cord blood stem cell samples, the plurality of cord blood stem cell banks including at least one private stem cell bank and at least one public stem cell bank;
identifying a plurality of facilities that will obtain cord blood stem cell samples using a uniform protocol;
using the uniform protocol to obtain cord blood stem cell samples at each of the plurality of facilities, the uniform protocol including the steps of:
providing, if a particular cord blood stem cell sample was previously identified for private storage at the at least one private stem cell bank, the particular cord blood stem cell sample to the at least one private stem cell bank for processing and storage;
determining, if the particular cord blood stem cell sample was not previously identified for private storage, whether characteristics of the mother or the particular cord blood stem cell sample match predetermined characteristics for cord blood stem cell samples required by the at least one public stem cell bank; and
supplying the particular cord blood stem cell sample to the at least one public stem cell bank for processing and storage if the characteristics of the mother or the particular cord blood stem cell sample match predetermined characteristics for cord blood stem cell samples required by the at least one public stem cell bank.

2. The method according to claim 1 wherein the step of determining includes the step of determining in advance of a birth that results in the particular cord blood stem cell sample whether characteristics of the mother match predetermined characteristics for cord blood stem cell samples required by the at least one public stem cell bank.

3. The method according to claim 2 wherein the step of determining further includes the step of determining at a birth that results in the particular cord blood stem cell sample whether characteristics of the particular cord blood stem cell sample match predetermined characteristics for cord blood stem cell samples required by the at least one public stem cell bank.

4. The method according to claim 1 wherein the step of determining further includes the step of determining at a birth that results in the particular cord blood stem cell sample whether characteristics of the particular cord blood stem cell sample match predetermined characteristics for cord blood stem cell samples required by the at least one public stem cell bank.

5. A method of collecting and distributing cord blood stem cells prior to storing, the method comprising the steps of:
identifying a plurality of cord blood stem cell banks that each have requirements for various different cord blood stem cell samples, the plurality of cord blood stem cell banks including at least one private stem cell bank and at least one public stem cell bank;
identifying a plurality of facilities that will obtain cord blood stem cell samples using a uniform protocol;
using the uniform protocol to obtain cord blood stem cell samples at each of the plurality of facilities, the uniform protocol including the steps of:
providing a networked computer system that includes a cord blood capture computer program, such that at each of the plurality of facilities there exists a computer that can execute the cord blood capture computer program;
marking, using the cord blood capture computer program, if a particular cord blood stem cell sample was previously identified for private storage at the at least one private stem cell bank, the marking including matching the particular cord blood stem cell sample with a code, and applying that code to an entry associated with the cord blood capture computer program that designates that particular cord blood stem cell sample with the at least one private stem cell bank;
determining, if the particular cord blood stem cell sample was not previously identified for private storage, whether characteristics of the mother or the cord blood stem cell sample match predetermined characteristics for cord blood stem cell samples required by the at least one public stem cell bank; and
marking, using the cord blood capture computer program and if the characteristics of the particular cord blood stem cell sample matched the predetermined characteristics, the particular cord blood sample for public storage at the at least one public stem cell bank, the marking including matching the particular cord blood stem cell sample with another code, and applying that another code to an entry associated with the cord blood capture computer program that designates that particular cord blood stem cell sample with the at least one public stem cell bank.

6. The method according to claim 5 wherein the step of determining includes the step of determining in advance of a birth that results in the particular cord blood stem cell sample whether characteristics of the mother match predetermined characteristics for cord blood stem cell samples required by the at least one public stem cell bank.

7. The method according to claim 6 wherein the step of determining further includes the step of determining at a birth that results in the particular cord blood stem cell sample whether characteristics of the particular cord blood stem cell sample match predetermined characteristics for cord blood stem cell samples required by the at least one public stem cell bank.

8. The method according to claim 5 wherein the step of determining further includes the step of determining at a birth that results in the particular cord blood stem cell sample whether characteristics of the particular cord blood stem cell sample match predetermined characteristics for cord blood stem cell samples required by the at least one public stem cell bank.
